(19) United States
(12) Patent Application Publication
(10) Pub. No.: US 2025/0019777 A1
(43) Pub. Date: Jan. 16, 2025

(54) ISOLATION, PURIFICATION, IN VITRO CULTIVATION, AND IDENTIFICATION OF SYMBIODINIUM SPECIES SYMBIOTIC WITH SOFT CORAL

(71) Applicant: Hainan Normal University, Haikou (CN)

(72) Inventors: Li WEI, Haikou (CN); Meng FENG, Haikou (CN); Han ZHU, Haikou (CN); Rong WANG, Haikou (CN); Xiangchen GAO, Haikou (CN); Jiecan YU, Haikou (CN)

(73) Assignee: Hainan Normal University, Haikou (CN)

(21) Appl. No.: 18/786,003

(22) Filed: Jul. 26, 2024

Related U.S. Application Data
(63) Continuation of application No. PCT/CN2023/134544, filed on Nov. 28, 2023.

(30) Foreign Application Priority Data
Jul. 13, 2023 (CN) 202310865383.8

(51) Int. Cl. C12Q 1/6895; C12N 1/12; C12Q 1/6844; C12R 1/89

(52) U.S. Cl. CPC C12Q 1/6895; C12N 1/125; C12Q 1/6844; C12Q 2600/13; C12R 2001/89

(57) ABSTRACT

The invention relates to isolation, purification, and in vitro cultivation of Symbiodinium algae symbiotic with soft corals. The deposit information for Symbiodinium sp. SY-1 is as follows: Depository Name: China General Microbiological Culture Collection Center (CGMCC); Depository Address: Institute of Microbiology, Chinese Academy of Sciences, Building 3, No. 1 Beichen West Road, Chaoyang District, Beijing, China; Deposit Date: Jun. 7, 2023; Deposit Number: CGMCC No. 40681; Taxonomy: Symbiodinium sp.

### CROSS-REFERENCE TO RELATED APPLICATION

This patent application is a national phase application of PCT application No. PCT/CN2023/134544, filed on Nov. 28, 2023, which claims the benefit and priority of Chinese Patent Application No. CN202310865383.8, entitled “ISOLATION, PURIFICATION, IN VITRO CULTIVATION, AND IDENTIFICATION OF SYMBIODINIUM SYMBIOTIC WITH SOFT CORAL”, and filed with the China National Intellectual Property Administration on Jul. 13, 2023, both of which are incorporated by reference herein in its entirety as part of the present application.

### REFERENCE TO SEQUENCE LISTING

A computer readable XML file entitled “GWPCTP20240503720_seqlist”, which was created on Jun. 20, 2024, with a file size of about 9,430 bytes, contains the sequence listing for this application, has been filed with this application, and is hereby incorporated by reference in its entirety.

### TECHNICAL FIELD

The present disclosure relates to the technical field of coral symbiotic algae in marine biology, in particular to a Symbiodinium species symbiotic with soft coral and methods for isolation, purification, in vitro cultivation, and morphological and molecular identification thereof.

### BACKGROUND

Coral reefs are essential components of marine ecosystems, providing a foundation for the diversity of marine life, much like tropical rainforests are crucial for terrestrial ecosystems. However, in recent years, coral mortality events have been escalating due to factors such as global warming. Since coral reefs are the direct product of coral growth metabolism, the death of corals will directly disrupt the ecosystem of coral reefs. One significant cause of coral mortality is coral bleaching. Coral polyps and photosynthetic zooxanthellae (Symbiodinium) form an endosymbiotic relationship. In this symbiotic relationship, the photosynthesis of Symbiodinium provides the energy foundation for the growth metabolism of corals. As corals themselves are transparent, the color of corals actually comes from the pigments of Symbiodinium. Coral bleaching is the process by which corals lose their endosymbiotic algae. Despite the extraordinary significance of the symbiotic relationship between corals and Symbiodinium, there is a lack of understanding of this relationship among people.

Zooxanthellae (Symbiodinium) are single-celled dinoflagellates that widely symbiotically inhabit marine invertebrates such as corals, clams, sponges, etc. In coral reef ecosystems, soft corals are one of the most fascinating inhabitants of the ocean, providing essential habitats for small fish, invertebrates, and algae on coral reefs. The mutualistic symbiotic relationship between Symbiodinium and corals is the foundation of coral reef ecosystems and an important part of coral physiological activities. The photosynthesis of Symbiodinium algae themselves can provide oxygen to corals, while the fixed carbon is transferred to the coral body to synthesize various substances needed for life activities. In return, corals provide protection to Symbiodinium and supply corresponding metabolic products as raw materials for photosynthesis. This cooperative relationship enables corals to better adapt to survival in oligotrophic waters. However, this symbiotic relationship is very fragile and can easily be disrupted. Minor environmental changes can lead to the loss of Symbiodinium or the destruction of pigments, causing coral bleaching and even death. Therefore, research on Symbiodinium has become an important part of coral research and conservation. However, due to the close symbiosis between Symbiodinium and corals in their natural state, related studies are difficult to conduct and are subject to various disturbances.

Symbiodinium consists of multiple clades. The A and E (Effrenium) clades that mainly symbiotically inhabit coral bodies are more extensively studied currently, and their isolation and large-scale in vitro cultivation have been achieved. However, knowledge of clades that widely symbiotically inhabit soft corals is still limited. By 2014, only one strain of coral endosymbiotic Symbiodinium had been successfully cultured in vitro globally, and this was limited to the laboratory cultivation stage. Therefore, by isolating and culturing Symbiodinium endosymbiotic within corals, the difficulty of zooxanthella-related research can be reduced, effectively facilitating studies on the physiological functions of Symbiodinium and the symbiotic relationship between corals and Symbiodinium, and providing a feasible approach to protect coral reef ecosystems, which is of great practical significance.

### SUMMARY

The present disclosure provides a Symbiodinium species symbiotic with soft coral and methods for isolation, purification, in vitro cultivation, and morphological and molecular identification thereof. It is an aim of the present disclosure to advance the understanding of Symbiodinium physiological functions, the symbiotic relationship between corals and Symbiodinium, and to provide a feasible approach to protect coral reef ecosystems.

In order to achieve the above objectives, the following technical solutions are provided.

In a first aspect, the present disclosure provides a Symbiodinium species symbiotic with soft coral, Symbiodinium sp. SY-1, where the deposit information for the Symbiodinium sp. SY-1 is as follows: Depository: China General Microbiological Culture Collection Center (CGMCC); Address: Institute of Microbiology, Chinese Academy of Sciences, Building 3, No. 1 Beichen West Road, Chaoyang District, Beijing 100101, China; Date of deposit: Jun. 7, 2023; Deposit number: CGMCC No. 40681; Taxonomy: Symbiodinium sp.

In a second aspect, the present disclosure provides use of the Symbiodinium sp. SY-1 in studying symbiotic relationship between corals and Symbiodinium in a coral reef system, coral physiology, and ecological function or conservation of Symbiodinium. The present disclosure further provides use of the Symbiodinium sp. SY-1 in the prevention and/or restoration of coral bleaching caused by ocean acidification.

In a third aspect, the present disclosure provides a method for isolating, purifying, and culturing the Symbiodinium sp. SY-1 in vitro, including:
step 1: obtaining soft coral tissue, cutting the soft coral tissue into small pieces with a sterile surgical blade, and mixing the small pieces with sterile seawater buffer to obtain an original suspension, where the soft coral tissue is collected from the Luhuitou Reef area in Sanya, Hainan Province, China;
step 2: examining the original suspension under a microscope to confirm presence of free single-celled algae;
step 3: filtering the original suspension through a 40 μm nylon mesh to remove tissue impurities while retaining nutrient-rich filtrate;
step 4: inoculating 1 mL of the nutrient-rich filtrate into a 24-well plate containing F/2 medium (with final antibiotic concentrations of 50 μg/mL kanamycin, 100 μg/mL ampicillin, 50 μg/mL streptomycin, and 2.5 μg/mL amphotericin B), and conducting culturing under 50 μmol/(m²·s) light intensity and 25° C. with gentle shaking at 30 r/min for 10-20 days to obtain algal culture of exponential growth phase; and
step 5: taking a pre-cultured algal culture containing coral tissue liquid, using approximately 200 μL for spreading on a solid medium (containing final antibiotic concentrations of 50 μg/mL kanamycin, 100 μg/mL ampicillin, 50 μg/mL streptomycin, and 2.5 μg/mL amphotericin B) for further isolation and purification to obtain pure Symbiodinium.

In a fourth aspect, the present disclosure provides a method for isolating, purifying, and culturing the Symbiodinium sp. SY-1 in vitro, including:
step 1: obtaining a soft coral tissue, cutting the soft coral tissue into small pieces with a sterile surgical blade, and mixing the small pieces with sterile seawater buffer to obtain an original suspension, where the soft coral tissue is collected from the Luhuitou Reef area in Sanya, Hainan Province, China;
step 2: examining the original suspension under a microscope to confirm presence of free single-celled algae;
step 3: filtering the original suspension through a 40 μm nylon mesh to remove tissue impurities while retaining nutrient-rich filtrate;
step 4: inoculating 1 mL of the nutrient-rich filtrate into a 24-well plate containing F/2 medium (with final antibiotic concentrations of 50 μg/mL kanamycin, 100 μg/mL ampicillin, 50 μg/mL streptomycin, and 2.5 μg/mL amphotericin B), and conducting culturing under 50 μmol/(m²·s) light intensity and 25° C. with gentle shaking at 30 r/min for 10-20 days to obtain algal culture of exponential growth phase;
step 5: taking the algal culture of exponential growth phase in step 4 for microscopy examination, identifying motile individuals with flagella, performing dilution separation by using a capillary to transfer individual algae into a 24-well plate containing 200 μL of F/2 medium, repeating the process at least 30 times to obtain at least 30 tubes of diluted and separated algae culture, obtaining single-celled algae, and conducting culturing in a 24-well plate;
step 6: placing the cultured 24-well plate under 50 μmol/(m²·s) light intensity at 25° C. for undisturbed culture for 10-20 days with gentle shaking at 30 r/min; examining microscopically for dividing cells and absence of protozoa; and
step 7: taking 200 μL of a culture solution in the 24-well plate, spreading the culture solution on a solid medium (containing final antibiotic concentrations of 50 μg/mL kanamycin, 100 μg/mL ampicillin, 50 μg/mL streptomycin, and 2.5 μg/mL amphotericin B) for further isolation and purification to obtain pure Symbiodinium sp. SY-1.

In a fifth aspect, the present disclosure provides a method for morphological and molecular identification of the Symbiodinium sp. SY-1, including:
step 1: obtaining a coral tissue, grinding the coral tissue in a buffer to obtain an original suspension;
step 2: microscopically examining the original suspension to confirm presence of free single-celled algae; and
step 3: inoculating the original suspension into F/2 medium for culturing to exponential growth phase, and then extracting DNA for PCR amplification of a molecular marker gene.

The Symbiodinium sp. SY-1, is a symbiotic algae (zooxanthella) within the soft coral (Platygyra sinensis) from the Luhuitou Reef area in Sanya, Hainan Province, China.

In the present disclosure, based on the characteristics of the symbiotic relationship between corals and Symbiodinium and their corresponding physiological mechanisms, the isolation, purification, and in vitro cultivation of Symbiodinium within coral bodies are carried out. In the initial stages of cultivation, impurities are not removed to ensure that the algae gradually adapts to the external cultivation environment. In the exponential growth phase, isolation and purification are performed to obtain pure reef-building symbiotic Symbiodinium, named Symbiodinium sp. SY-1. It is an aim of the present disclosure to advance the understanding of Symbiodinium physiological functions, and the symbiotic relationship between corals and Symbiodinium, and to provide a new algal species for the protection of coral reef ecosystems.

In the present disclosure, when the Symbiodinium sp. SY-1 is cultured in F/2 medium as the basic nutrient broth, the algal cells are spherical or oval (during division), with a diameter ranging from 8 to 12 μm. Each cell contains multiple chloroplasts, one nucleus, and one pyrenoid, with a cell wall of approximately 220 nm. Additionally, such microalga reproduces through binary fission and has a thick cell wall. Molecular identification confirms it belongs to the genus Symbiodinium. It exhibits prominent characteristics of wall attachment growth and phototropism during its growth process, surpassing other zooxanthella in the same genus. In terms of physiological characteristics, the alga shows significant acid tolerance and can grow normally at low PH levels (3-4). These characteristics are key focuses for future research, providing valuable scientific materials for studies on coral symbiosis, coral protection under ocean acidification, and potentially contributing to the recovery process of coral bleaching under ocean acidification.

Compared to existing technologies, the embodiments of the present disclosure have the following unique advantages. The Symbiodinium species symbiotic with soft coral, Symbiodinium sp. SY-1, exhibits significant differences in morphological characteristics and evolutionary aspects compared to previously reported coral symbiotic algae and other microalgal species. It displays stronger characteristics of wall attachment growth and phototropism compared to other zooxanthellae, making it a new algal species. The method of isolation and cultivation involved in this disclosure achieves the first reported purification and cultivation of Symbiodinium from soft corals in laboratory settings, providing excellent research materials for studying Symbiodinium functions and their symbiotic relationship with corals. This alga may also hold potential applications in coral artificial cultivation, coral reef ecosystem reconstruction, and novel algal materials.

### BRIEF DESCRIPTION OF THE DRAWINGS

To better illustrate the technical solutions in the embodiments or prior art used herein, a brief introduction of the drawings required in the examples will be provided as follows.

FIG. 1 is a photo of the soft coral sample collected from the Luhuitou Reef area in Sanya, Hainan Province, China;

FIG. 2 is a photo showing the plate streaking isolation of the Symbiodinium sp. SY-1;

FIG. 3 is a photo of a liquid culture of the Symbiodinium sp. SY-1;

FIG. 4 is a microscopic image of the Symbiodinium sp. SY-1 in exponential growth phase;

FIG. 5 is a scanning electron microscope (SEM) image of the Symbiodinium sp. SY-1 in the exponential growth phase;

FIG. 6 shows a transmission electron microscope (TEM) image of the Symbiodinium sp. SY-1 in the exponential growth phase;

FIG. 7 depicts an evolutionary tree (based on the 18S rRNA gene) of the Symbiodinium sp. SY-1;

FIG. 8 depicts an evolutionary tree (based on ITS2) of the Symbiodinium sp. SY-1; and

FIG. 9 is a photo showing the liquid culture of the Symbiodinium sp. SY-1 at pH values of 2-4.

### DEPOSITORY STATEMENTS

The Symbiodinium species symbiotic with soft coral, Symbiodinium sp. SY-1, was deposited at China General Microbiological Culture Collection Center (CGMCC) on Jun. 7, 2023, under the deposit number of CGMCC No. 40681. The deposit address is Building 3, No. 1 Beichen West Road, Chaoyang District, Beijing.

### DETAILED DESCRIPTION OF THE EMBODIMENTS

To further illustrate the present disclosure, the embodiments of the present disclosure will be described in detail below in conjunction with the accompanying drawings and examples, but they should not be construed as limiting the protection scope of the present disclosure.

### Examples of the Present Disclosure

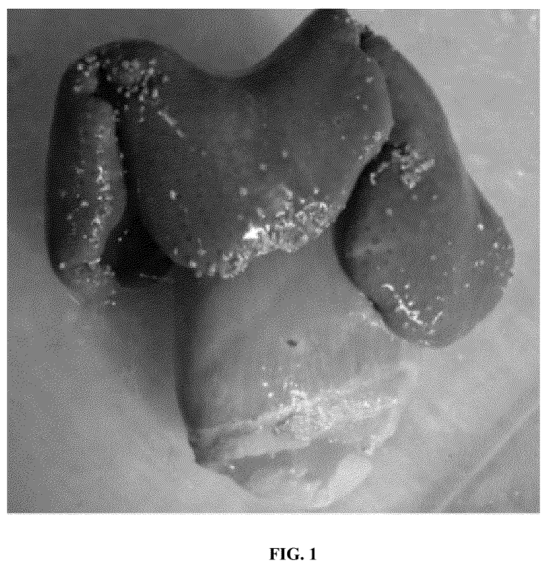

(1) Symbiodinium from soft coral (FIG. 1), which was collected by scuba diving in the Luhuitou Reef area, Sanya, Hainan, was isolated and cultured in vitro. Sterile seawater containing antibiotics and F/2 seawater medium were prepared in advance. The concentration of kanamycin in sterile seawater was 50 μg/mL, ampicillin was 100 μg/mL, streptomycin was 50 μg/mL, and amphotericin B was 2.5 μg/mL. The amount of sea salt in the F/2 seawater medium was 3%. The concentration of kanamycin in the F/2 seawater medium was 50 μg/mL, ampicillin was 100 μg/mL, streptomycin was 50 μg/mL, and amphotericin B was 2.5 μg/mL.

(2) A 5-cm² of soft coral tissue was placed in a sterilized mortar and washed 3 times with seawater filtered with a 0.22-μm filter. Sterile seawater was added, and the soft coral tissue was cut into small pieces using sterile scissors and ground with a grinding rod. Then, the mixture was filtered through a 40-μm sterile nylon mesh to remove impurities. The filtrate containing Symbiodinium cells was collected for subsequent preliminary culture operations.

(3) 1 mL of the filtrate containing algal cells was added to an F/2 medium containing antibiotics for preliminary culture. The concentrations of antibiotics in the F/2 medium were 50 μg/mL for kanamycin, 100 μg/mL for ampicillin, 50 μg/mL for streptomycin, and 2.5 μg/mL for amphotericin B. The culture temperature was set at 25° C., and the light intensity was 50 μmol/(m²·s).

(4) The filtrate containing algal cells was centrifuged at 1200×g for 5 min, and a resulting supernatant was discarded. The algal cell pellet was washed 3 times with an F/2 medium containing antibiotics to remove most of the impurities. The cell pellet was resuspended in the F/2 medium to prepare a cell suspension. The concentrations of antibiotics in the F/2 medium were 50 μg/mL for kanamycin, 100 μg/mL for ampicillin, 50 μg/mL for streptomycin, and 2.5 μg/mL for amphotericin B.

(5) 200 μL of the resuspended cell suspension was spread onto the F/2 solid medium containing antibiotics. The concentrations of antibiotics in the F/2 agar medium were 50 μg/mL for kanamycin, 100 μg/mL for ampicillin, 50 μg/mL for streptomycin, and 2.5 μg/mL for amphotericin B. The agar content in the F/2 solid medium was 0.8%.

(6) Capillary tubes were prepared by using an alcohol lamp to burn the tip of a glass Pasteur pipette and quickly pulling it to form a capillary tube with a diameter of about 20-50 μm. Symbiodinium cells cultured for several weeks in step 3 were observed under an inverted microscope, and viable Symbiodinium were selected. Under an inverted microscope, a single viable Symbiodinium cell was aspirated using a capillary tube and transferred to a new flask for culture.

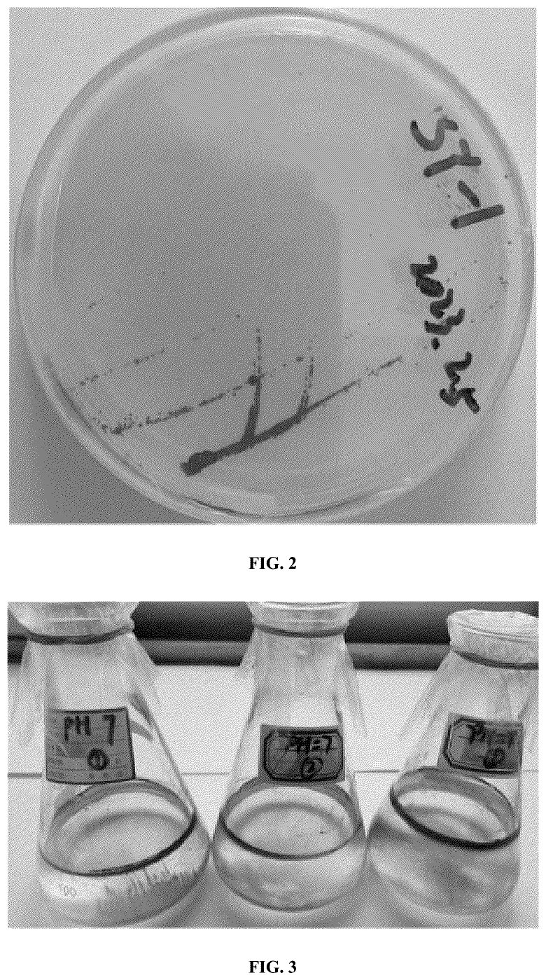

(7) From the algal colonies grown in step 5, single colonies were picked using a toothpick and streaked onto an F/2 medium containing 0.8% agar and antibiotics for plate streaking culture to obtain pure algal strains of Symbiodinium. After 2-3 consecutive transfers and purifications, the culture was gradually scaled up to conical flasks for subculture. Finally, single-clone cultured Symbiodinium was obtained (FIGS. 2 and 3). The F/2 medium was supplemented with 50 μg/mL kanamycin, 100 μg/mL ampicillin, 50 μg/mL streptomycin, and 2.5 μg/mL amphotericin B.

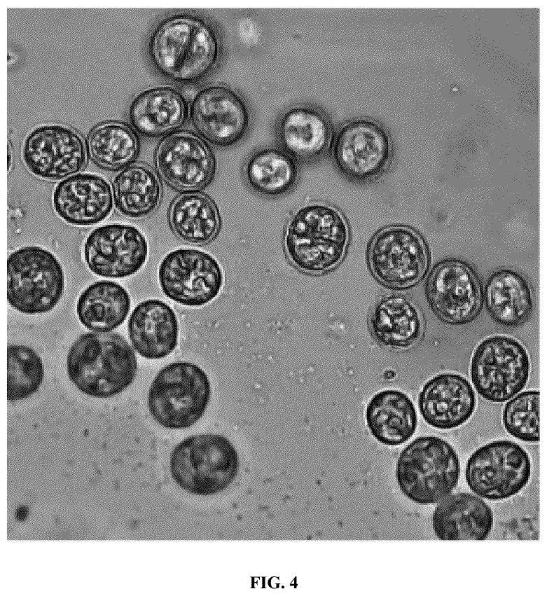

(8) Optical microscopy was carried out to observe the morphology of Symbiodinium isolated from soft coral. The morphology of Symbiodinium was observed using an inverted microscope (Nikon, CKX53), and the cell morphology was observed and photographed using an optical microscope (Nikon, ECLIPSE Ni-E). Images were processed using ImageJ, and cell size was measured. The cell size was calculated from 30 cells. Symbiodinium cells were observed to be spherical or ovoid with a diameter of 8-10 μm, and exhibited light yellowish-green color (FIG. 4).

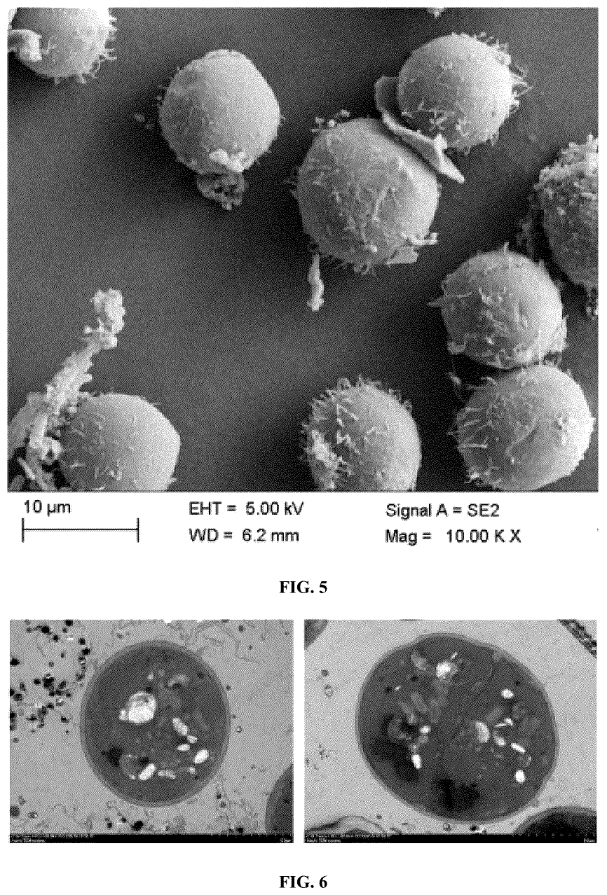

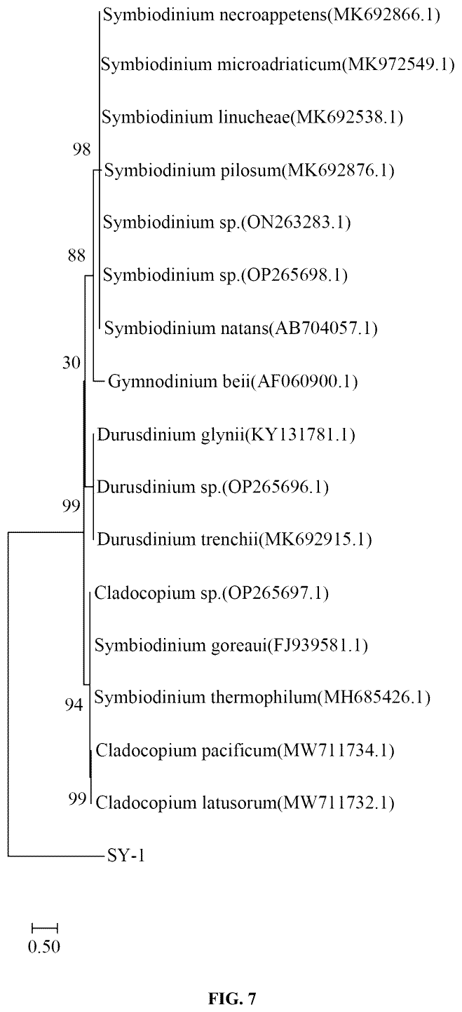

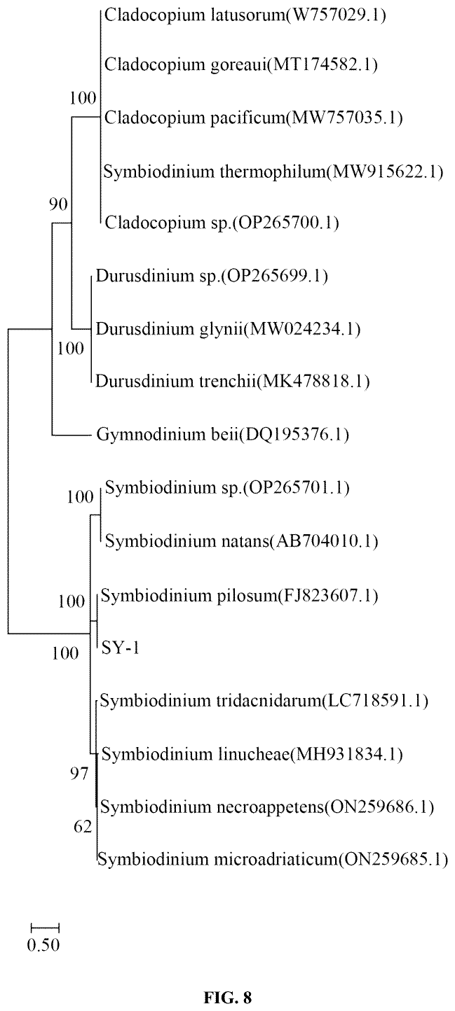

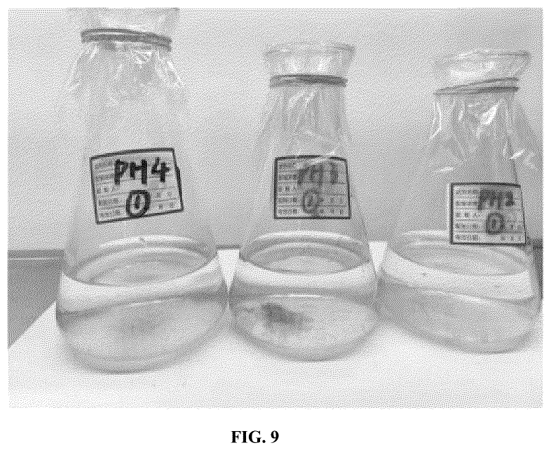

(9) SEM was employed to observe the morphology of Symbiodinium isolated from soft coral. Symbiodinium cells in the logarithmic growth phase were collected. During the logarithmic growth phase, the algal cells were collected by centrifugation of 50 mL of algal culture (horizontal rotor at 1500×g for 10 min) and washed twice with 0.1 mol/L phosphate-buffered saline (PBS, pH 7.4) for 10 min each. The cells were then centrifuged to obtain a pellet of at least the size of a mung bean. Glutaraldehyde-paraformaldehyde (2.0%-3.0%) (G1102-10 mL, Wuhan Servicebio Technology Co., Ltd.) was added, and the cells were fixed at room temperature in the dark for 2 h, then stored at 4° C. for later use. The fixed samples were washed 3 times with 0.1 M phosphate buffer (PB) (pH 7.4), 15 min each time. A 1% agar solution osmium tetroxide in 0.1 M PB (pH 7.4) was used for fixation at room temperature in the dark for 1-2 h. The samples were washed 3 times with 0.1 M PB (pH 7.4), 15 min each time. Then, the cells were transferred sequentially to 10%-20%-30%-40%-50%-60%-70%-80%-90%-95%-100% ethanol for 15 min each, and then to isoamyl acetate for 15 min. The samples were dried in a critical point dryer, attached to a double-sided conductive carbon adhesive and sputter-coated with gold on the sample stage of an ion sputtering instrument for about 30 s. Images were taken under a scanning electron microscope. The Symbiodinium cells were observed to be spherical or oval, with a diameter of 8-10 μm, and had peritrichous flagella (FIG. 5).

(10) TEM was used to observe the morphology of Symbiodinium isolated from soft coral. During the logarithmic growth phase, algal cells were collected by centrifuging 50 mL of algal culture (horizontal rotor at 1500×g for 10 minutes) to obtain the cells. The cells were washed twice with 0.1 mol/L PBS (pH 7.4) for 10 minutes each. The collected cell pellet should be at least the size of a mung bean. The cells were fixed with glutaraldehyde-polyoxymethylene (2.0%-3.0%) (G1102-10 mL, Wuhan Servicebio technology Co., Ltd.) in the dark at room temperature for 2 hours and stored at 4° C. for later use. The fixed samples were rinsed three times with 0.1 M PB (pH 7.4) for 15 minutes each. A 1% agar solution was melted by heating in advance, added to an EP tube, and allowed for cool. The suspension was picked and placed in the agar for embedding before agar coagulation. A 1% uranyl acetate solution in 0.1 M PB (pH 7.4) was prepared and used for fixation at room temperature in the dark for 2 hours. The samples were then washed three times with 0.1 M PB (pH 7.4) for 15 minutes each. The samples were dehydrated in a gradient of ethanol (30%, 50%, 70%, 80%, 95%, and two times 100%) for 20 minutes each, followed by embedding in SPI resin (812). The samples were embedded in a mixture of acetone and 812 resin (1:1) at 37° C. for 2-4 hours, followed by acetone and 812 resin (1:2) at 37° C. overnight, and pure 812 resin at 37° C. for 5-8 hours. The pure resin was poured into embedding molds, and the samples were inserted before curing at 37° C. overnight. The embedding molds were polymerized in a 60° C. oven for 48 hours, and the resin blocks were retrieved for further processing. The resin blocks were sectioned using an EM UC7 ultramicrotome (Leica) to obtain ultrathin sections, which were then picked up onto 150-mesh copper grids. The grids were stained with 2% uranyl acetate saturated alcohol solution in the dark for 8 minutes, followed by three washes in 70% alcohol, three washes in ultrapure water, and staining with 2.6% lead citrate solution for 8 minutes. The samples were rinsed three times in ultrapure water and gently dried on filter paper. Finally, the samples were observed and photographed using a HITACHI HT7800 transmission electron microscope. Each cell was observed to contain multiple chloroplasts, a single nucleus, and a pyrenoid, with a cell wall thickness of approximately 220 nm. Additionally, the microalgae reproduced through binary fission and exhibited thick cell walls (FIG. 6).

(11) To conduct molecular identification of Symbiodinium, single-clonal algal cultures were collected by centrifugation at 5000 rpm for 10 minutes. Genomic DNA of Symbiodinium was extracted following the instructions of the OMEGA Plant DNA Kit. The quality and purity of the extracted DNA were assessed using Qubit 4 and Nanoview. The extracted DNA was used as a template for polymerase chain reaction (PCR) to amplify the 18S rDNA, ITS, and Rbcl sequences for evolutionary analysis and molecular identification.

(12) For the amplification of the PCR 18S rDNA gene, PCRs were carried out in a 50 μL reaction system, including approximately 50 ng of DNA, 20 μL of TaKaRa Taq™ Version 2.0 plus dye PCR mix, 2 μL each of forward and reverse primers (10 μmol/L), and ddH₂O. The nuclear small subunit (nss) 18S rDNA gene fragment was amplified using the primers ss5 (SEQ ID NO: 2: 5′-GGTTGATCCTGCCAGTAGTCATATGCTTG-3′) and ss3 (SEQ ID NO: 3: 5′-AGCACTGCGTCAGTCCGAATAATTCACCGG-3′). The PCR program consisted of an initial denaturation at 94° C. for 5 minutes, followed by 30 cycles of denaturation at 94° C. for 1 minute, annealing at 55° C. for 2 minutes, extension at 72° C. for 3 minutes, and a final extension at 72° C. for 10 minutes. The PCR products were analyzed by 1% agarose gel electrophoresis and sequenced.

(13) Analysis based on ITS2 and LSU sequences

Using the extracted genomic DNA of Symbiodinium as a template, the internal transcribed spacer 2 (IT′S2) gene fragment was amplified by PCR using primers ITS intfor2 (SEQ ID NO: 4: 5′-GAATTGCAGAACTCCGTG-3′) and ITS2 reverse (SEQ ID NO: 5: 5′-GGGATCCATATGCTTAAGTTCAGCGGGT-3′). The PCR program included an initial denaturation at 94° C. for 5 minutes, followed by 30 cycles of denaturation at 94° C. for 30 seconds, annealing at 51° C. for 1 minute, extension at 72° C. for 30 seconds, and a final extension at 72° C. for 10 minutes.

(14) The large subunit (LSU) 28S rRNA gene fragment was amplified using primers 28S forward (SEQ ID NO: 6: 5′-CCCGCTGAATTTAAGCATATAAGTAAGCGG-3′) and 28S reverse (SEQ ID NO: 7: 5′-GTTAGACTCCTTGGTCCGTGTTTCAAGA-3′). The PCR program included an initial denaturation at 90° C. for 5 minutes, followed by 30 cycles of denaturation at 94° C. for 1 minute, annealing at 60° C. for 1 minute, extension at 72° C. for 1 minute, and a final extension at 72° C. for 5 minutes. The PCR products were subjected to 1% agarose gel electrophoresis and then sent to Sangon Biotech (Shanghai) Co., Ltd. for sequencing.

(15) Taxonomy identification and phylogenetic analysis.

The ITS2 genotype of the algal strain was compared with the non-redundant Symbiodinium ITS2 database (Sym-ITS2) (sym-its2.marinegenomics.cn), and combined with BLAST homology comparison results of ITS2 and LSU gene sequences from the National Center for Biotechnology Information (NCBI) database (blast.ncbi.nlm.nih.gov) for species confirmation of Symbiodinium. The software MEGA X was used to construct phylogenetic evolutionary trees based on the neighbor-joining (NJ) method using the Kimura 2-parameter (K2) model and the maximum likelihood (ML) method using the Hasegawa-Kishino-Yano (HKY) model. Bootstrap tests were performed with 1000 replicates to estimate the branch node confidence of the phylogenetic tree.

The nucleotide sequence of the 18S rRNA and ITS has a length of 1027 bp, and is set forth in SEQ ID NO: 1:

TGGTGATCCTGCCAGTAGTCATATGCTTGTCTCAAAGATTAAGCCATGC
ATGTCTCAGTATAAGCTTCTACACGGCGAAACTGCGAATGGCTCATTAA
AGCAGTTATAATTTATTTGATGGTCACTGCTACATGGATAACTGTGGTA
ATTCTAGAGCTAATACATGCACCAAAACCCAACTTCGCAGAAGGGTTGT
ATTTATTAGATACAGAACCATCGCAGGCTCTGCCTGGTTGTGGTGATTC
ATGATAACTCGATGAATCGTGTGGCTTGGCCGACGATGCATCTTTCAAG
TTTCTGACCTATCAGCTTCCGACGGTAGGGTATGGGCCTACCGTGGCAA
TGACGGGTAACGGAGAATTAGGGTTTGATTCCGGAGAGGGAGCCTGAGA
AACGGCTACCACATCTAAGGAAGGCAGCAGGCGCGCAAATTACCCAATC
CTGACACAGGGAGGTAGTGACAAGAAATAACAATACAGGGCATCCATGT
CTTGTAATTGGAATGAGTAGAATTTAAACCCCTTTATGAGTATCAATTG
GAGGGCAAGTCTGGTGCCAGCAGCCGCGGTAATTCCAGCTCCAATAGCG
TATATTAAAGTTGTTGCGGTTAAAAAGCTCGTAGTTGGATTTCTGTTGA
GGATGACCGGTCCGCCTTCTGGGTGTGTATCTGGCTCAGCCTTGACATC
TTCCTAAAGAACGTATCTGCACTTCATTGTGTGGTGCGGTATTTAGGAC
ATTTACCTTGAGGAAATTAGAGTGTTTCAAGCAAGCGATTGCCTTGAAT
ACATTAGCATGGAATAATAAGATAGGACCTCAGTTCTATTTTGTTGGTT
TCTAGAGCTGAGGTAATGGTCGATAGGGATAGTTGGGGGCATTCGTATT
TAACTGTCAGAGGTGAAATTCTTGGATTTGTTAAAGACGGACTACTGCG
AAAGCATTTGCCCAAGGGATGTTTTCATTGATCAGAACGAAAGTTAGGG
GATCGAGACGATCAGATACCGTCCTAGTCTTAACCCATAAAACTATG.

(16) The Symbiodinium sp. strain SY-1, isolated from soft corals, exhibited high acid tolerance. By comparing its growth at different pH levels, it was observed that the alga could grow at relatively low pH levels (even grew slowly at a minimum pH of 3; as shown in FIG. 9), indicating its adaptation to lower pH environments. With the increasing atmospheric CO2 concentration leading to ocean acidification, the coral reef ecosystem faces severe challenges. Due to its ability to withstand acidic conditions, this alga may potentially be used in the future for coral bleaching caused by ocean acidification. This involves reintroducing the Symbiodinium back to the coral to restore the survival and growth of corals under lower pH conditions, providing a possible solution for coral reef protection and ecological restoration.

In conclusion, this study achieved the laboratory purification and cultivation of a new species of coral-derived alga through techniques such as soft coral tissue pretreatment, short-term pre-cultivation of algae from coral sources, algal isolation and purification, and in vitro cultivation. The alga strain's morphological characteristics were observed using optical microscopy, SEM, and TEM. Furthermore, the full-length sequence of the nuclear small subunit ribosomal RNA gene (18S rRNA), internal transcribed spacer 2 (ITS2) sequence, and partial sequence of the ribulose-1,5-bisphosphate carboxylase/oxygenase gene (rbcL) were cloned. By comparing these genetic sequences and microscopic morphology with publicly available algal gene sequences, the alga strain was identified as a new species, designated as Symbiodinium sp. SY-1. The successful isolation and cultivation of this new species of coral-derived alga provide valuable research material for studying the functions of marine algae and their potential symbiotic relationships with corals. This alga may also have potential applications in coral artificial cultivation, coral reef reconstruction, and novel algal materials.

Although the above examples provide a detailed description of the present disclosure, they represent only a part of the embodiments of the invention, rather than all embodiments. Other embodiments falling within the scope of the invention can be obtained based on the embodiments disclosed herein without creative effort.

What is claimed is:

1. (canceled)

2. (canceled)

3. (canceled)

4. (canceled)

5. (canceled)

6. (canceled)

7. A method for isolating, purifying, and culturing a Symbiodinium sp. SY-1 in vitro, comprising:
step 1: obtaining soft coral tissue, cutting the soft coral tissue into small pieces with a sterile surgical blade, and mixing the small pieces with sterile seawater buffer to obtain an original suspension;
step 2: examining the original suspension under a microscope to confirm presence of free single-celled algae;
step 3: filtering the original suspension through a 40 μm nylon mesh to remove tissue impurities while retaining nutrient-rich filtrate;
step 4: inoculating 1 mL of the nutrient-rich filtrate into a 24-well plate containing F/2 medium, with final antibiotic concentrations of 50 μg/mL kanamycin, 100 μg/mL ampicillin, 50 μg/mL streptomycin, and 2.5 μg/mL amphotericin B, and conducting culturing under 50 μmol/m²/s light intensity and 25° C. with gentle shaking at 30 r/min for 10-20 days to obtain algal culture of exponential growth phase; and
step 5: taking a pre-cultured algal culture containing coral tissue liquid, using approximately 200 μL for spreading on a solid medium containing final antibiotic concentrations of 50 μg/mL kanamycin, 100 μg/mL ampicillin, 50 μg/mL streptomycin, and 2.5 μg/mL amphotericin B for further isolation and purification to obtain pure Symbiodinium;
wherein the Symbiodinium sp. SY-1 has a deposit number of CGMCC No. 40681 and is taxonomized to Symbiodinium sp.

8. A method for isolating, purifying, and culturing a Symbiodinium sp. SY-1 in vitro, comprising:
step 1: obtaining a soft coral tissue, cutting the soft coral tissue into small pieces with a sterile surgical blade, and mixing the small pieces with sterile seawater buffer to obtain an original suspension;
step 2: examining the original suspension under a microscope to confirm presence of free single-celled algae;
step 3: filtering the original suspension through a 40 μm nylon mesh to remove tissue impurities while retaining nutrient-rich filtrate;
step 4: inoculating 1 mL of the nutrient-rich filtrate into a 24-well plate containing F/2 medium, with final antibiotic concentrations of 50 μg/mL kanamycin, 100 μg/mL ampicillin, 50 μg/mL streptomycin, and 2.5 μg/mL amphotericin B, and conducting culturing under 50 μmol/m²/s light intensity and 25° C. with gentle shaking at 30 r/min for 10-20 days to obtain algal culture of exponential growth phase;
step 5: taking the algal culture of exponential growth phase in step 4 for microscopy examination, identifying motile individuals with flagella, performing dilution separation by using a capillary to transfer individual algae into a 24-well plate containing 200 μL of F/2 medium, repeating the process at least 30 times to obtain at least 30 tubes of diluted and separated algae culture, obtaining single-celled algae, and conducting culturing in a 24-well plate;
step 6: placing the cultured 24-well plate under 50 μmol/m²/s light intensity at 25° C. for undisturbed culture for 10-20 days with gentle shaking at 30 r/min; examining microscopically for dividing cells and absence of protozoa; and
step 7: taking 200 μL of a culture solution in the 24-well plate, spreading the culture solution on a solid medium containing final antibiotic concentrations of 50 μg/mL kanamycin, 100 μg/mL ampicillin, 50 μg/mL streptomycin, and 2.5 μg/mL amphotericin B for further isolation and purification to obtain pure Symbiodinium sp. SY-1;
wherein the Symbiodinium sp. SY-1 has a deposit number of CGMCC No. 40681 and is taxonomized to Symbiodinium sp.

9. A method for morphological and molecular identification of a Symbiodinium sp. SY-1, comprising:
step 1: obtaining a coral tissue, grinding the coral tissue in a buffer to obtain an original suspension;
step 2: microscopically examining the original suspension to confirm presence of free single-celled algae; and
step 3: inoculating the original suspension into F/2 medium for culturing to exponential growth phase, and then extracting DNA for PCR amplification of a molecular marker gene;
wherein the Symbiodinium sp. SY-1 has a deposit number of CGMCC No. 40681 and is taxonomized to Symbiodinium sp.


SEQUENCE LISTING (7 sequences)

SEQ ID NO: 1 - RNA, 1027 nt, Symbiodinium sp.
  source 1..1027: mol_type = genomic RNA; note = 18S rRNA and ITS
tggtgatcctgccagtagtcatatgcttgtctcaaagattaagccatgcatgtctcagtataagcttctacacggcgaaactgcgaatggctcattaaagcagttataatttatttgatggtcactgctacatggataactgtggtaattctagagctaatacatgcaccaaaacccaacttcgcagaagggttgtatttattagatacagaaccatcgcaggctctgcctggttgtggtgattcatgataactcgatgaatcgtgtggcttggccgacgatgcatctttcaagtttctgacctatcagcttccgacggtagggtatgggcctaccgtggcaatgacgggtaacggagaattagggtttgattccggagagggagcctgagaaacggctaccacatctaaggaaggcagcaggcgcgcaaattacccaatcctgacacagggaggtagtgacaagaaataacaatacagggcatccatgtcttgtaattggaatgagtagaatttaaacccctttatgagtatcaattggagggcaagtctggtgccagcagccgcggtaattccagctccaatagcgtatattaaagttgttgcggttaaaaagctcgtagttggatttctgttgaggatgaccggtccgccttctgggtgtgtatctggctcagccttgacatcttcctaaagaacgtatctgcacttcattgtgtggtgcggtatttaggacatttaccttgaggaaattagagtgtttcaagcaagcgattgccttgaatacattagcatggaataataagataggacctcagttctattttgttggtttctagagctgaggtaatggtcgatagggatagttgggggcattcgtatttaactgtcagaggtgaaattcttggatttgttaaagacggactactgcgaaagcatttgcccaagggatgttttcattgatcagaacgaaagttaggggatcgagacgatcagataccgtcctagtcttaacccataaaactatg

SEQ ID NO: 2 - DNA, 29 bp, synthetic construct
  source 1..29: mol_type = other DNA; note = primer ss5
ggttgatcctgccagtagtcatatgcttg

SEQ ID NO: 3 - DNA, 30 bp, synthetic construct
  source 1..30: mol_type = other DNA; note = primer ss3
agcactgcgtcagtccgaataattcaccgg

SEQ ID NO: 4 - DNA, 18 bp, synthetic construct
  source 1..18: mol_type = other DNA; note = primer ITS intfor2
gaattgcagaactccgtg

SEQ ID NO: 5 - DNA, 28 bp, synthetic construct
  source 1..28: mol_type = other DNA; note = primer ITS2 reverse
gggatccatatgcttaagttcagcgggt

SEQ ID NO: 6 - DNA, 30 bp, synthetic construct
  source 1..30: mol_type = other DNA; note = primer 28S forward
cccgctgaatttaagcatataagtaagcgg

SEQ ID NO: 7 - DNA, 28 bp, synthetic construct
  source 1..28: mol_type = other DNA; note = primer 28S reverse
gttagactccttggtccgtgtttcaaga